(19) United States
(12) Patent Application Publication
(10) Pub. No.: US 2020/0078268 A1
(43) Pub. Date: Mar. 12, 2020

(54) HIGH-SPEED SOFT CAPSULE FORMING MACHINE

(71) Applicant: In-Gyu CHOI, Suwon-si (KR)

(72) Inventor: In-Gyu CHOI, Suwon-si (KR)

(21) Appl. No.: 16/468,565

(22) PCT Filed: Jan. 22, 2018
(86) PCT No.: PCT/KR2018/000932

§ 371 Date: Jun. 11, 2019

(30) Foreign Application Priority Data
Jan. 23, 2017 (KR) 10-2017-0010346
Aug. 31, 2017 (KR) 10-2017-0110870
Nov. 3, 2017 (KR) 10-2017-0145650

(51) Int. Cl. A61J 3/07; B29C 51/14; B29C 65/70; A61K 9/48

(52) U.S. Cl. CPC A61J 3/07; B29C 51/14; B29C 65/70; B29C 2945/76531; B29C 2791/001; B29C 2791/002; A61K 9/4808

(57) ABSTRACT

Disclosed is a high-speed soft capsule forming machine comprising: a sheet forming part for forming two gelatin sheets; a pair of die rolls for heat sealing the gelatin sheets and simultaneously forming the same into a capsule shape; a wedge positioned at the upper sides of the pair of die rolls, heating the gelatin sheets at a predetermined temperature, and having a plurality of nozzles, which are formed to be aligned therein, for receiving liquid medicine from a liquid medicine feeding part and respectively injecting the same between the gelatin sheets inserted into cavities of the pair of die rolls; and a controller for controlling the rotational speed of the die rolls, the temperature of the wedge, the rotational speed and temperature of a cooling drum, and the intervals of nozzle operations for injecting the liquid medicine according to the angle of the nozzles formed in the wedge with respect to the center of the die roll and a set number value of lines in which the nozzles are aligned.

### TECHNICAL FIELD

The present invention relates to a forming machine which forms various liquid medicines together with gelatin into the form of a soft capsule, and more particularly to a high-speed soft capsule forming machine which can remarkably increase productivity through high-speed forming and can stably produce a soft capsule of excellent quality by minimizing defect rate.

### BACKGROUND ART

Nowadays, soft capsules obtained by filling raw liquid (powder, oil, or the like) of medicine in a capsule film mainly including an animal material such as gelatin or a vegetable material such as starch and sealing (enclosing) the capsule film have been known, and the soft capsules are generally manufactured by using rotary forming machines.

For example, a rotary forming machine suggested in Japanese Patent Application No. 1999-221267 includes sheet forming units for forming gelatin sheets of a thin film shape with a gelatin solution, a capsule forming unit for forming a capsule with the gelatin sheets, and a liquid medicine feeding unit for feeding liquid medicine into the encapsulated gelatin sheets.

In particular, the capsule forming unit includes a pair of die rolls which rotate in opposite directions while facing each other in a state in which cavities engraved into a one-half shape of the soft capsule correspond to the outer peripheries of the die rolls to form an outer sheath of the soft capsule, and a wedge for injecting (ejecting) liquid medicine is mounted on the upper sides of the die rolls and is connected to the liquid medicine feeding unit to receive the liquid medicine.

Accordingly, soft capsules in which raw liquid is filled in the gelatin films are continuously manufactured, by inserting two gelatin sheets formed by the sheet forming units between the pair of rotating die rolls of the capsule forming units and injecting the raw liquid between the gelatin sheets at a predetermined cycle at the same time, by heat-sealing the gelatin sheets and cutting the gelatin sheets at the same time, and by drawing the gelatin sheets into a capsule shape from the pair of die rolls.

In the manufactured method, since the cavities formed in the pair of facing die rolls to correspond to each other have to be precisely arranged such that the cavities coincide with each other and the soft capsule has to be formed in correspondence to the time line of the wedge in order to increase the quality of the soft capsule and reduce defect rate, the setting of the positions of the cavities is very important.

Meanwhile, as illustrated in FIGS. 1 and 2, according to the wedge 13 of the conventional capsule forming unit, liquid medicine discharge holes 13a are located to a height within 10 mm from a lower end of the wedge 13 to correspond to cavities 11b and 11b′ of the die rolls 11 and 11′, and nozzles 13b are formed at a height h2 that is higher than an end of the liquid medicine discharge holes 13a, that is, the lower end of the wedge 13 by 10 mm.

This provides a specific rotation time and a specific distance such that the gelatin sheets that constitute a soft capsule C are stably joined and sealed while leakage of the liquid medicine is minimized when the gelatin sheets are heat-sealed.

Further, pockets are formed in the gelatin sheets A and A′ by the pressure of the feeding pressure of the liquid medicine M and the cavities 11b and 11b′ of the die rolls 11 and 11′ at the moment when the liquid medicine M is fed due to the characteristics of the physical properties of the liquid medicine M, and since the gelatin sheets A and A′ are temporarily expanded and then are slightly shrunk in the process, the gelatin sheets A and A′ can be stably joined and sealed only when they are sealed after lapse of a predetermined time.

For example, when the diameter of the die rolls 11 and 11′ is 150 mm, thirty cavities 11b and 11b′ are formed on the outer periphery of each of the die rolls 11 and 11′, and the diameter of the cooling drums of the sheet forming unit is 500 mm, a time of a minimum of 0.58 seconds and a maximum of 0.63 seconds has to elapse until the contraction of the liquid medicine is finished after the liquid medicine is discharged from the nozzles 13b, and then, the gelatin sheets A and A′ can be joined and sealed excellently only when the temperature of the gelatin sheets A and A′ is maintained at 39° C. to 40° C.

That is, the conventional soft capsule forming machine has a limit in that productivity significantly deteriorates due to a structural operation restriction, for example, of having to maintain the rotational speed of the die rolls 11 and 11′ at 2.0 RPM/min or less or (intermittently) rotate the die rolls 11 and 11′ again after temporarily stopping the die rolls 11 and 11′ and to set the height h2 of the nozzles 13b to 10 mm or less in order to satisfy the quality condition while minimizing the defects at the joining portion and the loss of the material and reduce noise generated by the operation of a piston (plunger) when the liquid medicine M is fed and injected.

Further, the sheet forming unit forms the gelatin solution into the gelatin sheets of the gelled thin films by using the spreader boxes and the cooling drums, and cools the gelatin sheets to a temperature of 18 to 22° C. to maintain the gel state of the gelatin sheets and cause the gelatin sheets to be easily separated from the cooling drums while feeding the gelatin sheets to the capsule forming unit, and the wedge 13 of the capsule forming unit heats the gelatin sheets A and A′ to a temperature of 38 to 45° C. such that the gelatin sheets A and A′ are instantaneously smoothly expanded and shrunk again by the pressure of the liquid medicine M, and then, the temperature of the die rolls 11 and 11′ is maintained at 24±2° C.

Accordingly, because a time for which the gelatin sheets A and A′ pass through the wedge 13 becomes shorter if production speed is made higher by making the rotational speed of the die rolls 11 and 11′ higher, the gelatin sheets A and A′ cannot be heated in a normal temperature condition for instantaneously smoothly expanding and shrinking the gelatin sheets A and A′, making the joining area (width and thickness) of the gelatin sheets A and A′ irregular so that the joining and cutting of the gelatin sheets A and A′ cannot be properly made and thus a defect, for example, of easily bursting the gelatin sheets A and A′ occurs.

If only the heating temperature of the wedge 13 is increased while the rotational speed of the die rolls 11 and 11′ is made higher, the gelatin sheets A and A′ receives high heat in a short time and are melt to be deflected downwards or become mushy so that the gelatin sheets A and A′ cannot be expanded and shrunk normally when the liquid medicine is fed and may be easily stuck to the die rolls 11 and 11′, causing a defect in sealing and cutting.

Further, malfunctions, abnormalities, and defect rate can be minimized only when the content of moisture in the gelatin sheets A and A′ fed to the capsule forming unit has to be maintained at a degree of 25 to 45%.

That is, if the content of the moisture in the gelatin sheets A and A′ is less than 25%, the gelatin sheets A and A′ may be wound on the outer peripheries of the die rolls 11 and 11′ and a separate mangle roller, and if the content of the moisture is more than 45%, defects in which the joining area of the capsule becomes smaller and thinner may occur, causing high defect rate.

Meanwhile, materials reacting with the animal gelatin cannot be contained as contents because the animal gelatin corresponds to animal proteins, almost all of which are extracted from domestic animals, such as cows or pigs, the animal gelatin may cause side-effects such as allergy after ingestion thereof, and diseases, such as bovine spongiform encephalopathy and the foot and mouth disease, which are infected to the human beings, and the use of the animal gelatin is avoided by Muslims for the reason of halal and haram.

Accordingly, as an alternative, a capsule formed by mixing a vegetable material, such as modified starch or carrageenan, with glycerin, purified water, and the like has been suggested, but it is difficult to manufacture the capsule because the physical properties of the vegetable gelatin are easily changed as compared with the animal gelatin.

That is, the animal gelatin is stably dried, that is, gelled even if the temperature thereof is rapidly lowered due to the lower viscosity, but the vegetable gelatin cannot properly form a pocket because the physical properties thereof change if the temperature difference is abruptly occurs due to the high viscosity or the form of the capsule cannot be maintained to be deformed or the capsule may burst as a crack is caused.

Here, it is noted that the prior art or the conventional technology is information carried by the user or obtained in the process of deriving the present invention, and is provided to help understanding of the technical meanings of the present invention and does not mean the technology widely known in the art to which the present invention pertains before the filing date of the present invention.

### DETAILED DESCRIPTION OF THE INVENTION

### Technical Problem

Accordingly, in comprehensive consideration of all the above-described items, the present inventor(s) has conceived of the present invention while making various efforts to develop a soft capsule forming machine of a new structure, which can achieve an effect of remarkably increasing productivity by making the forming speed, the cooling and heating temperatures of gelatin sheets, and an operation interval of a liquid medicine feeding unit for injecting liquid medicine with nozzles different according to an angle from a starting point, which is a section from the center of die rolls to a part in which the gelatin sheets are joined and sealed, to the nozzles formed in a wedge and the number of rows of the arranged nozzles to prevent changes of the physical properties and smoothly perform operations thereof, thereby minimizing the defect rate and the loss of the materials and stably forming a soft capsule of an excellent quality at a high speed, can minimize the defect rate and the loss of the materials due to a time for which the liquid medicine is discharged from the nozzles and erroneous setting of the rotation ratio of the die rolls interworking the nozzles, and can prevent the changes of the physical properties by making the heating temperatures of the gelatin sheets, which are necessary when the capsule is formed, different according to the content of the moisture of the gelatin sheets, and reducing an abrupt change range, after consistently studying the soft capsule forming machine, while making an emphasis on solving the limits and problems of the existing soft capsule forming machine.

Accordingly, a technical problem that is to be solved by the present invention and a purpose of the present invention are to provide a high-speed soft capsule forming machine which can stably form a soft capsule of an excellent quality at a high speed while minimizing defect rate and loss of materials.

Further, another technical problem that is to be solved by the present invention and another purpose of the present invention are to provide a high-speed soft capsule forming machine which can minimize defect rate even if an error is generated in operation times and intervals of nozzles and die rolls.

Further, another technical problem that is to be solved by the present invention and another purpose of the present invention are to provide a high-speed soft capsule forming machine which can allow high-speed forming of a soft capsule, thereby remarkably increasing productivity and being able to be used in general purpose regardless of the kind of gelatin.

Here, the technical problems that are to be solved by the present invention and the purposes of the present invention are not limited to the above-mentioned technical problems and purposes, and the other technical problems and purposes, which have not been mentioned, will be understood more clearly from the following description by an ordinary person skilled in the art.

### Technical Solution

A detailed means according to a first implementation of the first invention for achieving the purposes of the present invention, which have been described above, and solving the technical problems of the present invention suggests a high-speed soft capsule forming machine including sheet forming units configured to coat a gelatin solution on cooling drums, which are rotating, to form two gelatin sheets, a pair of die rolls having cavities engraved into a one-half shape of the capsule provided on outer peripheries of the pair of die rolls so as to rotate in opposite directions while facing each other and approaching each other in a state in which the cavities are arranged to correspond to each other, in order to form the gelatin sheets into a capsule shape while heat-sealing the gelatin sheets, and a wedge which is located above the pair of die rolls, in which a plurality of nozzles for receiving liquid medicine from a liquid medicine feeding unit and injecting the liquid medicine between the gelatin sheets inserted into the cavities of the pair of die rolls are arranged in a zigzag form, and in which heaters for heating the gelatin sheets to a predetermined temperature are embedded in the wedge; and provided with a controller configured to adjust forming speed by controlling a rotational speed of the pair of die rolls, a temperature of the wedge, a rotational speed and a temperature of the cooling drums, and an operation interval for injecting the liquid medicine with the nozzles, according to an angle of the nozzles formed in the wedge with respect to the centers of the pair of die rolls and a preset number of rows of the arranged nozzles.

Accordingly, according to the first implementation of the present invention, productivity can be remarkably increased by making the forming speed, the cooling and heating temperatures of gelatin sheets, and an operation interval of a liquid medicine feeding unit for injecting liquid medicine with nozzles different according to an angle from a starting point, which is a section from the center of die rolls to a part in which the gelatin sheets are joined and sealed, to the nozzles formed in a wedge and the number of rows of the arranged nozzles to prevent changes of the physical properties and smoothly perform operations thereof, thereby minimizing the defect rate and the loss of the materials and stably forming a soft capsule of an excellent quality at a high speed.

Further, according to the first implementation of the present invention, since the controller controls such that whenever an angle from a starting point, which is a section from the centers of the pair of die rolls 20 to a part in which the gelatin sheets S are joined and sealed, to the nozzles formed in the wedge increases by 30° from a reference angle (30°), the rotational speed of the pair of die rolls increases by 4 to 6 RPM/min from a reference rotational speed (4 to 6 RPM/min), a surface temperature of the wedge increases by 1 to 2° C. from a reference temperature (38 to 42° C.), the rotational speed of the cooling drums increases by 1.2 to 1.8 RPM/min from a reference rotational speed (1.2 to 1.8 RPM/min), a surface temperature of the cooling drums decreases by 1° C. from a reference temperature (20 to 18° C.), and the interval for the operations of the nozzles for one dose of liquid medicine further increases by 3 times per second from a reference number of times (3 times per second), and when the number of rows of the nozzles increases by 1 row from a reference row (1 row) formed in the reference angle, the number of times decreases by a value obtained by dividing the reference number of times for the reference angles by the increased number of rows, soft capsules of an excellent quality can be stably and efficiently produced while defect rate is minimized, by heating and cooling the gelatin sheets into a stable state without any change in temperature, thereby preventing change of the physical properties.

Further, according to the first implementation of the present invention, since one or more rows of nozzles are arranged along concave surfaces of lower portions of the wedge formed to correspond to the pair of die rolls, and are symmetrically arranged on opposite sides of the wedge to be located within an angle range of 7° to 168° from the center of one die roll, soft capsules of an excellent quality can be stably produced by properly adjusting a temperature, which is necessary for cooling and heating of the gelatin sheets, according to the forming speed of the capsules.

Further, according to the first implementation of the present invention, since curved protrusions which heat the gelatin sheets while contacting the gelatin sheets protrude convexly from the opposite sides of the wedge, defect rate can be minimized by heating the gelatin sheets more stably.

A detailed means according to a second implementation of the present invention suggests a high-speed soft capsule forming machine including sheet forming units configured to form two gelatin sheets from a gelatin solution, a pair of die rolls having cavities engraved into a one-half shape of the capsule provided on outer peripheries of the pair of die rolls so as to rotate in opposite directions while facing each other and approaching each other in a state in which the cavities are arranged to correspond to each other, in order to form the gelatin sheets into a capsule shape while heat-sealing the gelatin sheets, and a wedge which is located on the upper side of the pair of die rolls, in which a pair of nozzles for receiving liquid medicine from a liquid medicine feeding unit and injecting the liquid medicine between the gelatin sheets, and in which heaters for heating the gelatin sheets to a predetermined temperature and a temperature sensor for detecting temperature are embedded, wherein a recess is formed long in a widthwise direction of the cavities to eject the liquid medicine to one cavity and another adjacent cavity at the same time after decreasing an ejection pressure of the liquid medicine, at an edge of a part of the wedge, in which the nozzle ejects the liquid medicine.

Accordingly, according to the second implementation of the present invention, since the liquid medicine is naturally injected into the pockets (cavities) of the gelatin sheets in a state in which the pressure of the liquid medicine is decreased by the recesses even if an error is generated in the operation times and intervals of the nozzles and the die rolls, the defect of the joining part due to an injection position error of the nozzles, and the loss of the materials can be minimized unlike the conventional technologies.

Further, according to the second implementation of the present invention, since a temperature sensor for detecting a temperature of the wedge is provided, a moisture sensor for electrically detecting a content of moisture in the gelatin solution is provided on each of the sheet forming unit, and a controller for adjusting a heating temperature of the wedge by controlling the heaters according to a detected moisture content of the moisture sensor and a detected temperature of the temperature sensor is provided, soft capsules of an excellent quality can be stably produced by making the heating temperatures, which are necessary when the capsules are formed, different according to the content of moisture in the gelatin sheets, and the soft capsule forming machine can be used in general purpose regardless of the kind of the gelation.

Further, according to the second implementation of the present invention, since the high-speed soft capsule forming machine includes preheating units configured to preheat the gelatin sheets into a stable state without any abrupt change of temperature, between the sheet forming units and the wedge, high-speed forming is possible so that defect rate can be minimized while productivity is remarkably increased.

Further, according to the second implementation of the present invention, since a moisture sensor for electrically detecting a content of moisture in the gelatin solution is provided on each of the sheet forming unit, and a controller for adjusting heating temperatures of the wedge and the preheating units by controlling the heaters of the wedge and the preheating units according to a detected moisture content of the moisture sensor is provided, a change in the physical properties of the gelatin sheets can be prevented by heating the gelatin sheets according to the content of moisture in the gelatin sheets in a stable state without any abrupt change in temperature so that soft capsules of an excellent quality can be stably produced while defect rate is minimized and the soft capsule forming machine can be used in general purpose regardless of the kind of the gelatin.

Further, according to the second implementation of the present invention, since the high-speed soft capsule forming machine may further include a water jacket formed on the outer side of the wedge to circulate cooling water, a cooler configured to circulate the cooling water in the water jacket through a cooling water inlet and a cooling water outlet of the water jacket and cool the cooling water which has absorbed heat while circulating the interior of the water jacket, and a controller configured to, if the detected temperature of the temperature sensor is a normal value or more, rapidly decrease the temperature of the wedge by controlling the cooler, soft capsules of an excellent quality can be stably produced by controlling the heating temperature of the wedge more efficiently.

Further, according to the second implementation of the present invention, since the controller controls such that a difference value from a reference of 25% is calculated if the content of moisture in the gelatin solution on the sheet forming units is less than 25% and the temperatures of the heaters of the wedge are decreased by the calculated difference value, and a difference value from a reference of 45% is calculated if the content of moisture is more than 45% and the temperatures of the heaters of the wedge are increased by the calculated difference value, soft capsules of an excellent quality can be produced stably and efficiently by controlling the heating temperature of the wedge more effectively.

### Advantageous Effects of the Invention

According to a first implementation of the present invention having the means and configurations for achieving the purposes and solving the technical problems, a change of the physical properties of gelatin sheets can be prevented and a soft capsule forming machine can be smoothly operated, by controlling such that the forming speed, the cooling and heating temperatures of gelatin sheets, and an operation interval of a liquid medicine feeding unit for injecting liquid medicine with nozzles are made different according to an angle from a starting point, which is a section from the center of die rolls to a part in which the gelatin sheets are joined and sealed, to the nozzles formed in a wedge and the number of rows of the arranged nozzles, and accordingly, liquid medicine can be injected naturally and stably into pockets (cavities) of the gelatin sheets while a defect of a joining and sealing part and the loss of the materials are minimized, and productivity can be remarkably enhanced by stably forming the soft capsules of an excellent quality at a high speed of a minimum of 3 times as compared with the existing technology.

Further, according to the first implementation of the present invention, since the number of rows of the nozzles arranged in a zigzag form can be increased to a maximum of four rows, noise can be minimized by reducing the operation numbers of a cap and a piston for feeding and injecting liquid medicine.

Further, according to a second implementation of the present invention, since the liquid medicine is naturally injected into the pockets (cavities) of the gelatin sheets in a state in which the pressure of the liquid medicine is decreased by the recesses even if an error is generated in the operation times and intervals of the nozzles and the die rolls, the defect of the joining part due to an injection position error of the nozzles, and the loss of the materials can be minimized unlike the conventional technologies.

Accordingly, since soft capsules of an excellent quality can be formed at a high speed of a minimum of 3 times as compared with the conventional technologies, productivity can be remarkably enhanced.

Further, according to another second implementation of the present invention, since a change of the physical properties of the gelatin sheets can be prevented by controlling the temperatures of the heaters and the preheating units embedded in the wedge according to the content of the moisture in the gelatin solution to heat the gelatin sheets in a stable state without any change in temperature, defect rate can be minimized while productivity is remarkably increased, and the capsule forming machine can be used in general purpose regardless of viscosity, temperature, drying degree (the degree of moisture that is necessary to form the gelatin sheets into a capsule and maintain the encapsulation) due to the material and kind of the gelatin.

Here, the aspect of the present invention is not limited thereto, and other unmentioned aspects of the present invention may be clearly appreciated by those skilled in the art from the following descriptions.

### DESCRIPTION OF THE DRAWINGS

FIGS. 1 and 2 are diagrams schematically illustrating a capsule forming unit of a soft capsule forming machine according to a conventional technology.

FIG. 3 is a diagram schematically illustrating a high-speed soft capsule forming machine according to a first embodiment of the present invention.

FIG. 4 is a cross-sectional view schematically illustrating a part (capsule forming unit) of the high-speed soft capsule forming machine according to the first embodiment of the present invention.

FIG. 5 is an enlarged cross-sectional view of a local portion of FIG. 4.

FIG. 6 is a circuit diagram schematically illustrating a configuration of a heater of a wedge of the high-speed soft capsule forming machine according to the first embodiment of the present invention.

FIGS. 7 to 11 are flowcharts for controlling an operation of the high-speed soft capsule forming machine according to the first embodiment of the present invention.

FIG. 12 is a diagram schematically illustrating a high-speed soft capsule forming machine according to a second embodiment of the present invention.

FIG. 13 is a sectional view schematically illustrating a part (capsule forming unit) of the high-speed soft capsule forming machine according to the second embodiment of the present invention.

FIG. 14 is an enlarged sectional view illustrating a die roll and a wedge of the high-speed soft capsule forming machine according to the second embodiment of the present invention when the die roll and the wedge are normally operated.

FIG. 15 is an enlarged sectional view illustrating a die roll and a wedge of the high-speed soft capsule forming machine according to the second embodiment of the present invention when the die roll and the wedge are abnormally operated.

FIG. 16 is a sectional view schematically illustrating a part (capsule forming unit) of a soft capsule forming machine according to another second embodiment of the present invention.

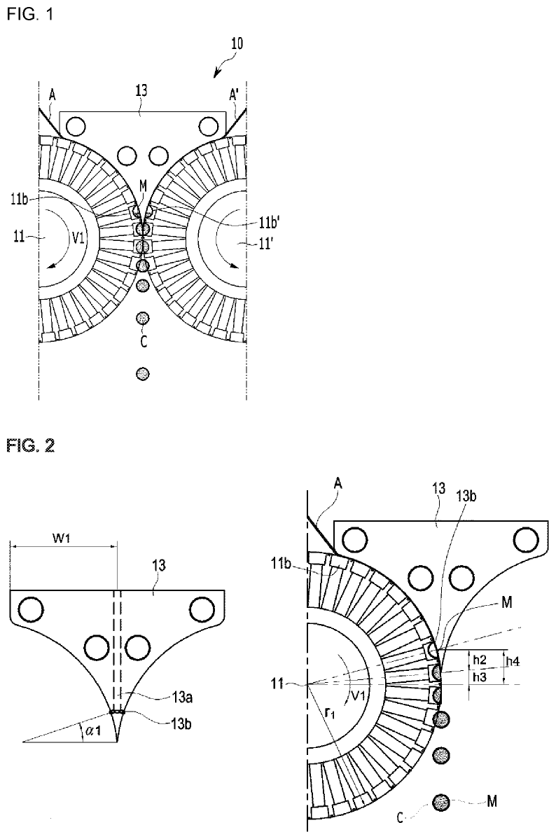

### BEST MODE

Hereinafter, exemplary embodiments of the present invention will be described in more detail with reference to the accompanying drawings.

Prior to the description of the present invention, it is explicitly noted that the terms, which will be described below, are defined in consideration of the functions of the terms in the present invention, and should be construed to have the concepts in conformity with the technical spirit of the present invention and the meanings generally used or commonly recognized in the art to which the present invention pertains.

Further, a detailed description of known functions or configurations related to the present invention will be omitted to avoid making the subject matter of the present invention unclear.

Here, in the accompanying drawings, elements are partially exaggerated or simplified for convenience and clarify of the description and understanding of the configurations and operations of the technology, and the sizes and forms of the elements in the drawings do not exactly coincide with the actual sizes and forms.

Further, when it is described that a part includes an element, it may mean that the part may further include second element without excluding the second element unless a specially contradictory description is made.

In addition, the terms ‘part’ and ‘unit’ mean the unit for processing at least one function or operation or the form of a module for performing some functions, and the part or the unit may be realized with a means, for example, through hardware, software, or a combination of hardware and software.

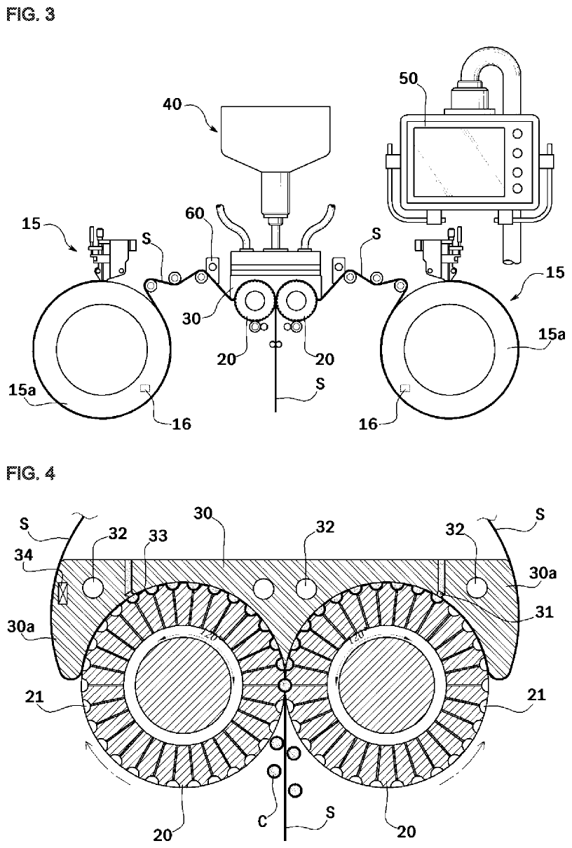

As illustrated in FIGS. 3 to 5, a high-speed soft capsule forming machine according to a first embodiment of the present invention largely includes sheet forming units 15, a pair of die rolls 20, a wedge 30, a liquid medicine feeding unit 40, and a controller 50.

That is, the high-speed soft capsule forming machine according to the first embodiment of the present invention forms two film type gelatin sheets S with a gelatin solution in the sheet forming units 15, heat-seals the film type gelatin sheets S in the pair of die rolls 20, which are rotating, and the wedge 30 and cuts the gelatin sheets S while injecting liquid medicine F fed from the liquid medicine feeding unit 40, between the gelatin sheets S at a predetermined cycle, and draws the gelatin sheets S into capsule shapes, in order to continuously manufacture soft capsules in which the liquid medicine F is filled inside the gelatin sheets S.

The sheet forming units 15 are parts which continuously form two thin film-shaped gelatin sheets S with the gelatin solution and transfer and feed the gelatin sheets S between the pair of die rolls 20 and the wedge 30, and two sheet forming units 15 are provided on the left and right sides of the pair of die rolls 20 and the wedge 30.

Here, each of the sheet forming units 15 may employ a general structure including a solution feed part, a spreader box, a cooling drum 15a, and a winding roller.

That is, the sheet forming units 15 may be configured to continuously form two thin film-shaped gelatin sheets S by coating the gelatin solution on the pair of left and right cooling drums 15a, which are rotating.

Moreover, a first temperature sensor 16 which detects a change of the temperature of the cooling drum and generates an electrical signal is provided on each of the sheet forming units 15.

The pair of die rolls 20 and the wedge 30 are parts which form a soft capsule by injecting and charging a predetermined amount of liquid medicine F fed from the liquid medicine feeding units 40, between the two gelatin sheets S transferred and fed from the sheet forming units 15 and cutting the gelatin sheets S, and are located at a middle part of the sheet forming units 15 for encapsulation.

Further, in order to form the two gelatin sheets S transferred and fed from the sheet forming units 15 into a capsule shape while heat-sealing the gelatin sheets S, cavities 21 engraved into a one-half shape of the capsule are provided on outer peripheries of the pair of die rolls 20 to rotate in opposite directions while facing each other and approaching each other in a state in which they are arranged to correspond to each other.

Here, blades for cutting off the encapsulated gelatin sheets S are formed at edges of the cavities 21.

Further, a hole for pulling the gelatin sheet S through vacuum absorption (negative pressure) or a suction operation or allowing a pocket to be naturally formed by a pressure at the moment when the liquid medicine F is ejected from the nozzle 31 is formed at a center of a bottom surface of each of the cavities 21.

The wedge 30 is located above the pair of die rolls 20, and at least one row of nozzles (31) for injecting the liquid medicine F between the gelatin sheets S inserted into the cavities of the pair of die rolls 20 are formed at a lower portion of the wedge 30 to correspond to the cavities 21 of the die rolls 20.

That is, the wedge 30 has a wedge shape in which concave surfaces are symmetrically formed on opposite sides of a lower portion thereof such that the pair of die rolls 20 are rotated without interference, one or more nozzles 31 which receive the liquid medicine F from the liquid medicine feeding unit 40 along the rotational axis direction of the die rolls 20 and inject the liquid medicine F between the encapsulated gelatin sheets S are symmetrically arranged, and a reference row (first row) of the nozzle 31 is arranged within a range of 7° to 150° from the center of one die roll 20.

Further, curved protrusion 30a, which heat the gelatin sheets S after allowing the gelatin sheets S to be adhered to each other while contacting each other, protrude convexly on opposite sides of the wedge 30.

That is, the curved protrusions 30a are formed to allow the gelatin sheets S transferred and fed from the sheet forming units 15 to be sent between the pair of die rolls 20 and the wedge 30 after heating the gelatin sheets S in advance while contacting the gelatin sheets S in a stable state without an abrupt change of temperature.

Liquid medicine feeding holes for connecting the nozzles 31 and the liquid medicine feeding unit 40 are formed on the upper surface of the wedge 30, and heaters 32 for heating the gelatin sheets S to a predetermined temperature and a second temperature sensor 34 for detecting the temperature of the wedge 30 are embedded in the wedge 30.

That is, as the heaters 32 heat the wedge 30 under the control of the controller 50, the heat that is necessary for heat-sealing is transferred to the gelatin sheet S.

Here, the wedge 30 has a shape which surrounds at least two fifths of the upper sides of the pair of die rolls 20, and thus, it is preferable that the reference row (first row) of the nozzles 31 are located within an angle range of 7° to 150° from the center of one die roll 20, or a plurality of rows of die rolls 20 are arranged at an interval of 4° to 6° from the reference row.

That is, the area of the wedge 30, which surrounds the upper sides of the pair of die rolls 20, may become larger as the formation locations of the nozzles 31 become more distant from the location of the encapsulation, and the rotational speeds of the die rolls 20 may be doubled according to the formation locations of the nozzles 31.

For example, if the diameters of the cooling drums 15a of the sheet forming units are 500 mm and the nozzles 31 are formed at locations of 30° on the curved surface of the wedge 30 while taking lines from the centers of the rotary shafts of the die rolls 30 having a diameter of 150 mm and having 30 cavities 21 at outer peripheries thereof to a part in which the gelatin sheets S are joined and sealed, as a starting point, an increase of about 3 times in speed may be possible as compared with the nozzle located at an existing angle of 7°, an increase of about 6 times in speed may be possible as compared with the nozzle located at an existing angle of 7° if the nozzles 31 are formed at a location of 60°, and an increase of about 9 times in speed may be possible as compared with the nozzle located at an existing angle of 7° if the nozzles 31 are formed at a location of 90°.

That is, whenever the angles of the nozzles 31 formed in the wedge 30 from the centers of the pair of die rolls 20 increase by 30° from the reference angle while a minimum joining time (a time until the two gelatin sheets are joined and sealed after the liquid medicine is injected between the gelatin sheets), for which the gelatin sheets S may be stably joined and sealed, is constantly maintained in a range of 0.58 seconds to 0.63 seconds, the rotational speeds of the die rolls 20 may increase 4 to 6 RPM/min from a reference rotational speed.

Moreover, when a plurality of reference rows of nozzles 31 are arranged in an angle range of 7° to 150° from the center of one die roll 20, the nozzles 31 may be formed at an interval corresponding to the arrangement interval of the cavities 21 of the die roll, and a plurality of rows of injection holes for feeding the liquid medicine to the nozzle 31 may be arranged to smoothly feed the liquid medicine.

For example, the several number of rows of nozzles 31 may be formed in parallel in one row to correspond to the cavities 21 of the die rolls along the curved surfaces of the lower side of the wedge 30, and preferably, may be zigzag nozzles formed or installed in the form of zigzags.

Further, when the nozzles 31 are zigzag nozzles, two or more rows of nozzles 31 may be arranged, and when the two or more rows of nozzles 31 are arranged, the interval from the reference row (first row) to the next row (second row) or the interval from the next row (second row) to the following row (third row) may be an angle of about 4 degrees to six degrees.

Further, the number of rows of the nozzles 31 may be differently determined according to the state of the liquid medicine, such as the form of powder, or the form of liquid or oil.

The liquid medicine feeding unit 40 is a part which feeds the liquid medicine between the gelatin sheets S, which are to be encapsulated, and is located above the wedge 30 to feed the liquid medicine toward the nozzles 31 of the wedge 30 at a predetermined cycle.

Here, the liquid medicine feeding unit 40 may employ a general structure including a hopper, a plunger type pump, and a tube assembly.

Since the liquid medicine feeding unit 40 may feed and inject the liquid medicine toward the several cavities 21 at the same time when a plurality of rows of nozzles 31 are formed in the wedge 30, operation noise and vibration may be minimized as the number of operations of a cam and a piston decreases.

Meanwhile, the viscosities of liquid medicine are different according to the kinds of the liquid medicine and in particular, the viscosities are much more different according to whether the liquid medicine are in the form of only oil or in the form of a mixture of oil and powder, and the liquid medicine is neither properly suctioned nor stably discharged due to high pressure if the viscosity of the liquid medicine is high in the process of pumping the liquid medicine with a plunger type pump of the liquid medicine feeding unit 40, and as a result, the feed of the liquid medicine through the nozzle 31 cannot be smoothly made and noise and vibration are severe so that the number of the rows of the nozzles 31 is inevitably restricted according to the kind of the liquid medicine.

For example, a limit value of the viscosity of the liquid medicine which may be formed when the angle from a starting point, which is a section from the center of the die roll 20 to a part in which the gelatin sheets S are joined and sealed, to the nozzle 31 formed in the wedge 30 is a reference angle of 30°, at which the reference row of nozzles 31 are formed, and the liquid medicine includes only an oil component is 5500 cp, a limit value of the viscosity of the liquid medicine which may be formed when the liquid medicine includes a mixture of oil and powder components is 3300 cp, a limit value of the viscosity of the liquid medicine which may be formed when the reference angle is 150°, at which the reference row of nozzles are formed, and the liquid medicine includes only an oil component is 3000 cp, and a limit value of the viscosity of the liquid medicine which may be formed when the liquid medicine includes a mixture of oil and powder components is 2100 cp.

That is, as an angle from a starting point, which is a section from the center of the die roll 20 to a part in which the gelatin sheets S are joined and sealed, to the nozzle 31 formed in the wedge 30 increases, the rotational speed of the die roll 20 becomes higher, and then, if the viscosity of the liquid medicine is high, the liquid medicine cannot be properly fed and injected toward the several cavities 21.

The controller 50 controls an overall operation of the forming machine, and in particular, adjusts forming speed by controlling the rotational speed of the die rolls 20, the temperature of the heater 32 of the wedge, the rotational speed and the temperature of the cooling drums 15a, and an interval of operations for injecting the liquid medicine by the nozzles 31 according to the angle of the nozzles 31 formed in the wedge 30 from the center of the pair of die rolls 20 and the preset number of rows of the arranged nozzles 31.

That is, the controller 50 adjusts the forming speed by controlling the rotational speeds of the die rolls 20 and the cooling drums 15a in proportion to the preset angles of the nozzles 31 from the centers of the pair of die rolls 20 and controlling the temperatures of the wedge 30 and the cooling drums 15a in inverse proportion to the preset angles of the nozzles 31 from the centers of the pair of die rolls 20.

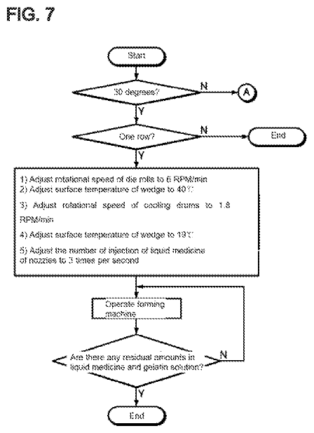

For example, as illustrated in FIGS. 7 to 11, whenever the angle from the starting point, which is a section from the center of the die roll 20 to a part in which the gelatin sheets S are joined and sealed, to the nozzle 31 formed in the wedge 30 increases by 30° from 30°, the controller 50 controls such that the rotational speed of the die rolls 20 increases by 4 to 6 RPM/min from a reference rotational speed of 4 to 6 RPM/min, the surface temperature of the wedge 30 increases by 1 to 2° C. from a reference temperature of 38 to 42° C., the rotational speed of the cooling drums 15a increases by 1.2 to 1.8 RPM/min from a reference rotational speed of 1.2 to 1.8 RPM/min, and the surface temperature of the cooling drums 15a decreases by 1° C. from a reference temperature of 20 to 18° C.

Then, the surface temperature of the cooling drums 15a is adjusted by controlling the temperature of the cooling drums 15a according to a detected temperature value of a first temperature sensor 16, the surface temperature of the wedge 30 is adjusted by controlling the heater 32 according to a detected temperature value of a second temperature sensor 34, and the rotational speed of the die rolls 20 and the rotational speed of the cooling drums 15a are adjusted by controlling the driving units, such as motors, for transmitting power.

Moreover, the controller 50 adjusts the forming speed by controlling the interval of the operations of the liquid medicine feeding unit 40 for injection of the liquid medicine by the nozzle 31 in proportion to the preset number of the rows of the arranged nozzles 31.

That is, the interval of the operations for injection of one dose of the liquid medicine by the nozzle 31 increases by 3 times per second whenever the angle from the starting point, which is a section from the center of the die roll 20 to a part in which the gelatin sheets S are joined and sealed, to the nozzle 31 formed in the wedge 30 increases by 30° from the reference angle (30°), and decreases by a value obtained by dividing the reference numbers for the reference angles by the increased number of rows whenever the number of rows of the nozzles 31 increases by one row from the reference row (one row) while the rotational speed of the die rolls 20 is the same.

For example, the reference row (first row) of the nozzles 31 are formed on 30° of the wedge 30 while a section from the center of the die roll 20 to a part in which the gelatin sheets S are joined and sealed is taken as the starting point, and the operations of the nozzles 31 are controlled to inject the liquid medicine 3 times per second when the number of the rows is one row.

Then, since the liquid medicine cannot be properly fed toward several cavities 21 if the viscosity of the liquid medicine is high, the operations of the nozzles 31 are controlled such that the liquid medicine is injected only when the number of rows of the nozzles 31 is one if a reference angle from a starting point, which is a section from the center of the die roll 20 to a part in which the gelatin sheets S are joined and sealed, to the nozzles 31 formed in the wedge 30 is 30 degrees, the operations of the nozzles 31 are controlled such that the liquid medicine is injected only when the number of rows of the nozzles 31 is one (reference row) or two if the reference angle is 60 degrees, and the operations of the nozzles 31 are controlled such that the liquid medicine is injected only when the number of rows of the nozzles 31 is two if the reference angle is 90 degrees.

In particular, the operations of the nozzles 31 are controlled such that the liquid medicine is injected only when the number of rows of the nozzles 31 is two (reference row) or four while the liquid medicine is limited to one which is not in the form of a mixture of oil and powder if the reference angle is 120 degrees, and the operations of the nozzles 31 is controlled such that the medicine is injected only when the number of rows of the nozzles 31 is four while the liquid medicine is limited to one which is not in the form of a mixture of oil and powder if the reference angle is 150 degrees.

Further, as illustrated in FIG. 6, the controller 50 may include a variable adjustor 37 for arbitrarily setting the temperature of the heaters 32, a microcomputer 38 configured to compare signals of the second temperature sensor 34 and the variable adjustor 37 and interrupt supply of a power source, a relay 39 installed on an electric wire for supplying a power source to control an on/off operation of the applied power source, and a transistor 39a configured to drive the relay 39.

That is, the microcomputer 38 maintains a temperature set by the variable adjustor 37 by recognizing the temperature set by the variable adjustor 37 and receiving a signal of the second temperature sensor 34, and comparing the recognized temperature and the signal to control the power source applied to the heater 32, and the microcomputer 38 interrupts the power source by sending a signal to the transistor 39a to turn off the relay 39 when the temperature detected by the second temperature sensor 34 is higher than the temperature set by the variable adjustor 37, and consistently applies the power source to the heaters of the wedge 32 to generate heat by sending a signal to the transistor 39a to maintain the relay 39 in an on state when the temperature detected by the second temperature sensor 34 is lower than the temperature set by the variable adjustor 37.

Meanwhile, the technology for controlling the surface temperature of the cooling drums 15a according to the detected temperature value of the first temperature sensor 16 by the controller 50 may employ a configuration of maintaining the surface temperature of the cooling drums 15 at a predetermined temperature by controlling the temperature of the cooling liquid (cooling water) to a predetermined temperature and circulating the cooling liquid in the interiors of the cooling drums 15a, and this technology is disclosed in patent document 1 and thus a detailed description thereof will be omitted.

The main operations and operational principles of the high-speed soft capsule forming machine according to the first embodiment of the present invention will be described below.

First, as illustrated in FIGS. 3 to 5, two gelatin sheets S are fed between the pair of die rolls 20 and the liquid medicine F is continuously ejected at a specific time interval through the two nozzles 31 of the wedge 30 located above the pair of die rolls 20 so that the liquid medicine F is injected between the two gelatin sheets S inserted between the die rolls 20. At the same time, the wedge 30 increases bonding force by heating the gelatin sheets S with the heaters 32 and dissolving the gelatin sheets S with the heat, and the pair of left and right die rolls 20 heat-seals the gelatin sheets S with a pressure formed when the gelatin sheets S pass through the minimum distance portion between the pair of die rolls 20. Then, the gelatin sheets S are cut to have a capsule shape as the blades (not illustrated) formed at edges of the cavities 21 of the die rolls 20 are engaged with each other while facing each other.

Moreover, mangle rollers are disposed on the lower side of the die rolls 20, which have completely formed a capsule so that the remaining gelatin sheets (scrap sheets) that are left after the capsule is cut off are pulled to the lower side and at the same time the soft capsule and the remaining gelatin sheets are separated from the gelatin sheets by release rollers, and only the soft capsule is retrieved via s shooter disposed on the front side. Then, the rotational speed of the mangle rollers is adjusted such that the remaining gelatin sheets are neither torn while being fed nor wound into the die rolls 20.

In this process, the controller 50 may transport and feed the gelatin sheets S naturally and smoothly while cooling and heating the gelatin sheets S by receiving the temperatures of the cooling drums 15a and the wedge 30 according to the forming speed of the capsule and automatically controlling them to predetermined values.

For example, as illustrated in FIG. 7, if the reference angle from the centers of the die rolls 20 to the nozzles 31 formed in the wedge 30 is 30° and the number of rows of the nozzles 31 is one (one row in a zigzag pattern), the controller 50 controls such that the rotational speed of the die rolls 20 is adjusted to 4 to 6 RPM/min, the reference temperature of the wedge 30 is adjusted to 38 to 42° C., the rotational speed of the cooling drums 15a is adjusted to 1.2 to 1.8 RPM/min, and the surface of the cooling drums 15a is adjusted to 20 to 18° C., and controls such that the operations of the nozzles 31 for injection the liquid medicine by 3 times are made in one seconds. That is, it is controlled such that the operation interval for injecting the liquid medicine one time is 0.333 seconds.

Then, it is controlled such that the step proceeds to step A if the reference angle is not 30°, and the operations of the nozzles 31 are stopped by determining the state as an error if the number of rows of the nozzles 31 is more than one (row).

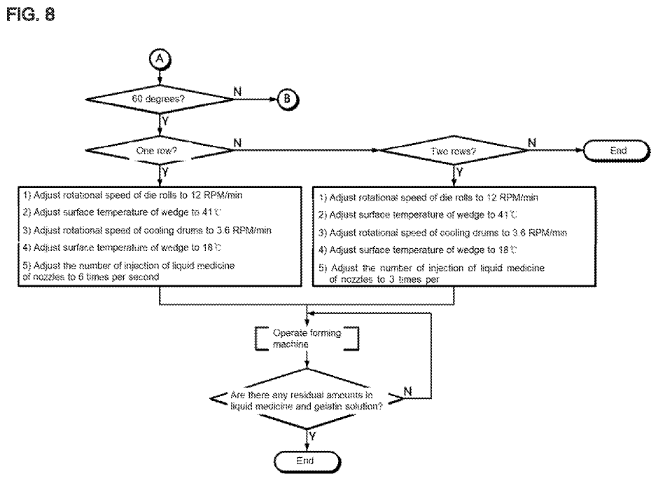

Further, as illustrated in FIG. 8, if the reference angle from the centers of the die rolls 20 to the nozzles 31 formed in the wedge 30 is 60° and the number of rows of the nozzles 31 is one, the controller 50 controls such that the rotational speed of the die rolls 20 is adjusted to 10 to 12 RPM/min, the reference temperature of the wedge 30 is adjusted to 39 to 43° C., the rotational speed of the cooling drums 15a is adjusted to 3.0 to 3.6 RPM/min, and the surface of the cooling drums 15a is adjusted to 19 to 17° C., and controls such that the operations of the nozzles 31 for injection the liquid medicine 6 times are made in one seconds. That is, it is controlled such that the operation interval for injecting the liquid medicine one time is 0.1666 seconds.

Then, the step proceeds to step B if the reference angle is not 60°, it is controlled such that the operation interval of the nozzles 31 for injecting the liquid medicine 3 times is one second if the number of rows of the nozzles 31 is not one (row) and is two (two rows in a zigzag pattern), and it is controlled such that the state is determined as an error and the operations of the nozzles 31 are stopped if the number of rows of the nozzles 31 is more than two (rows).

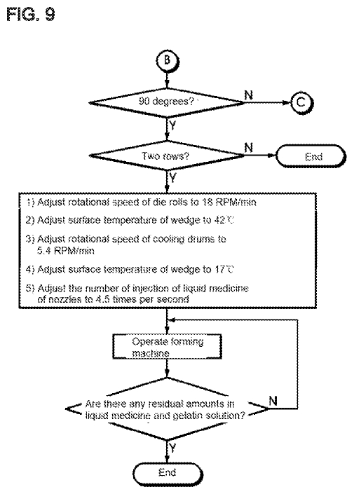

Further, as illustrated in FIG. 9, if the reference angle from the centers of the die rolls 20 to the nozzles 31 formed in the wedge 30 is 90° and the number of rows of the nozzles 31 is two (rows), the controller 50 controls such that the rotational speed of the die rolls 20 is adjusted to 16 to 20 RPM/min, the reference temperature of the wedge 30 is adjusted to 40 to 44° C., the rotational speed of the cooling drums 15a is adjusted to 4.8 to 5.4 RPM/min, and the surface of the cooling drums 15a is adjusted to 18 to 16° C., and controls such that the operations of the nozzles 31 for injecting the liquid medicine 4.5 times are made in one seconds. That is, it is controlled such that the operational interval for injecting the liquid medicine one time is 0.2222 seconds.

Then, it is controlled such that the step moves to step C if the reference angle is not 90°, and the operations of the nozzles 31 are stopped by determining the state as an error if the number of rows of the nozzles 31 is less than two (rows) or more than two rows (two rows in a zigzag pattern).

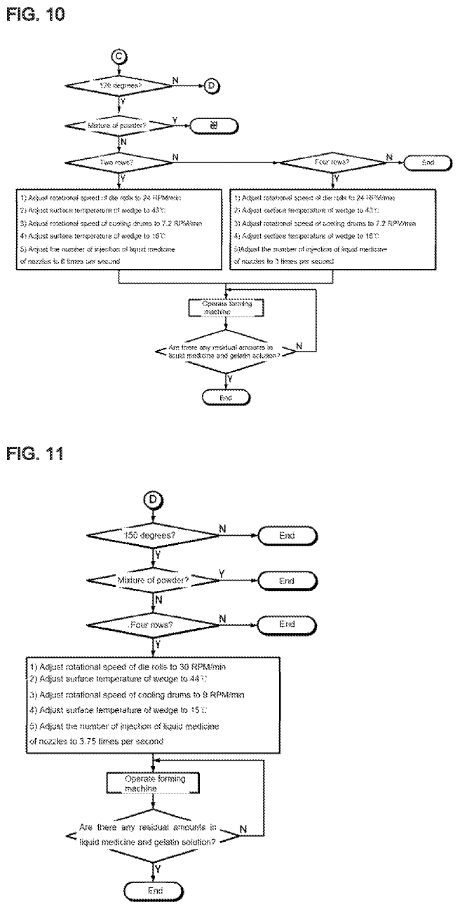

Further, as illustrated in FIG. 10, if the reference angle from the centers of the die rolls 20 to the nozzles 31 formed in the wedge 30 is 120° and the number of rows of the nozzles 31 is two (two rows in a zigzag pattern), the controller 50 controls such that the rotational speed of the die rolls 20 is adjusted to 22 to 26 RPM/min, the reference temperature of the wedge 30 is adjusted to 41 to 45° C., the rotational speed of the cooling drums 15a is adjusted to 6.6 to 7.2 RPM/min, and the surface of the cooling drums 15a is adjusted to 17 to 15° C., and controls such that the operations of the nozzles 31 for injecting the liquid medicine 6 times are made in one seconds. That is, it is controlled such that the operational interval for injecting the liquid medicine one time is 0.1666 seconds.

Then, the step proceeds to step D if the reference angle is not 120°, it is controlled such that the operation interval of the nozzles 31 for injecting the liquid medicine 3 times is one second if the number of rows of the nozzles 31 is not two (two rows in a zigzag pattern) but is four (four rows in a zigzag pattern), and it is controlled such that the state is determined as an error and the operations of the nozzles 31 are stopped if the number of rows of the nozzles 31 is less than two (rows) and more than four (rows).

Further, if the liquid medicine input by a vision device which measures and inspects the component of the liquid medicine and the like is determined to be the form in which oil and powder are mixed, the state is determined to be an error and it is controlled such that the operations of the nozzles 31 are stopped.

Further, as illustrated in FIG. 11, if the reference angle from the centers of the die rolls 20 to the nozzles 31 formed in the wedge 30 is 150° and the number of rows of the nozzles 31 is four (four rows in a zigzag pattern), the controller 50 controls such that the rotational speed of the die rolls 20 is adjusted to 28 to 32 RPM/min, the reference temperature of the wedge 30 is adjusted to 42 to 46° C., the rotational speed of the cooling drums 15a is adjusted to 8.4 to 9 RPM/min, and the surface of the cooling drums 15a is adjusted to 16 to 14° C., and controls such that the operations of the nozzles 31 for injecting the liquid medicine 3.75 times are made in one seconds. That is, it is controlled such that the operational interval for injecting the liquid medicine one time is 0.266 seconds.

Then, it is controlled such that the operations of the nozzles 31 are stopped by determining the state as an error if the reference angle is not 150° or the number of rows of the nozzles 31 is less than or more than four (rows).

Further, if the liquid medicine input by a vision device which measures and inspects the component of the liquid medicine and the like is determined to be the form in which oil and powder are mixed, the state is determined to be an error and it is controlled such that the operations of the nozzles 31 are stopped.

The high-speed soft capsule forming machine according to the first embodiment of the present invention may increase production rate by about 3 times as compared with an existing capsule forming machine in which the nozzles are located at 7°, the rotational speed of the die rolls is 2 RPM/min, the reference temperature of the wedge 30 is 39° C., and the rotational speed and the surface temperature of the cooling drums 15a are 0.6 RPM/min and 18° C., for example, because the diameter of the cooling drums 15a of the sheet forming unit is 500 mm, and the nozzles 31 are formed at locations of 30° with respect to the rotary shafts of the die rolls 20, the diameter of which is 150 mm and which has 30 cavities 21 on the outer peripheries thereof.

Accordingly, since the inferiorities of the jointing parts and the loss of the material due to the changes in the physical properties of the gelatin sheets S during a high-speed forming operation may be minimized and a high-speed forming operation of soft capsules of excellent quality may be stably performed a minimum of 3 times or more as compared with the existing technology, productivity can be remarkably improved.

In other words, if production speed is increased by making the rotational speeds of the die rolls 20 and the cooling drums 15a higher for high-speed forming of capsules, a time for which the gelatin sheets S are cooled by the cooling drums 15a becomes shorter so that capsules cannot be properly formed and a time for which the gelatin sheets S pass through the wedge 30 becomes shorter so that the gelatin sheets S cannot be heated in a normal temperature condition in which the gelatin sheets are instantaneously smoothly expanded and then contracted when the liquid medicine is fed, and accordingly, the joining area (width and thickness) between the gelatin sheets S is not uniform so that the gelatin sheets S cannot be properly joined and cut off, causing an error of bursting easily.

Accordingly, the high-speed soft capsule forming machine can be used in general purpose regardless of forming speed, by the controller 50 automatically controlling such that the rotational speed of the die rolls 20 and the cooling drums 15a and the heating temperature of the cooling drums 15a and the heaters 32 embedded in the wedge, and the operation interval of the liquid medicine feeding unit 40 for injection of the liquid medicine by the nozzles 31 are different according to an angle from a starting point, which is a section from the center of the die roll 20 to a part in which the gelatin sheets S are joined and sealed, to the nozzles 31 formed in the wedge 30 and the number of rows of the arranged nozzles 31 to prevent changes in the physical properties of the gelatin sheets S by cooling and heating the gelatin sheets S in a stable state without any abrupt change in temperature so that defect rate can be minimized and operation stop time can be reduced while productivity is remarkably increased.

Moreover, a plurality of rows of nozzles 31 arranged in a zigzag form may be formed so that noise due to frequent driving can be minimized by reducing the number of operations of the cam and the piston (plunger) of the liquid medicine feeding unit 40 for feeding and injecting the liquid medicine.

### MODES FOR CARRYING OUT THE INVENTION

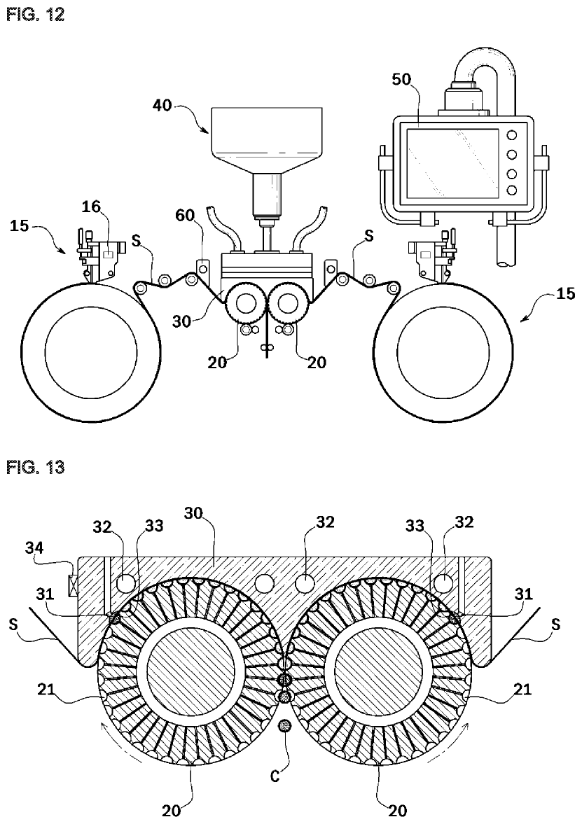

As illustrated in FIGS. 12 to 15, a high-speed soft capsule forming machine according to a second embodiment of the present invention largely includes sheet forming units 15, a pair of die rolls 20, a wedge 30, a liquid medicine feeding unit 40, a controller 50, and a preheating unit 60.

That is, the sheet forming units 15 form two film type gelatin sheets S with a gelatin solution, heats and seals the film type gelatin sheets S in the pair of die rolls 20, which are rotating, and the wedge 30 and at the same time, cuts the gelatin sheets S while injecting liquid medicine F fed from the liquid medicine feeding unit 40, between the gelatin sheets S at a predetermined cycle, and draws the gelatin sheets S into capsule shapes, in order to continuously manufacture soft capsules in which the liquid medicine F is filled inside the gelatin sheets S.

The sheet forming units 15 are parts which continuously form gelatin sheets S of a thin film with a gelatin solution and transport and feed the gelatin sheets S, and are provided on the left and right sides of the pair of die rolls 20 and the wedge 30.

Further, a moisture sensor 16 for electrically detecting moisture in the gelatin solution is provided on each of the sheet forming units 15.

Here, each of the sheet forming units 15 may employ a general structure including a solution feeding part, a spreader box, a forming drum, and a winding roller.

In this case, the moisture sensor 16 may be mounted on the solution feeding part or the spreader box.

The pair of die rolls 20 and the wedge 30 are parts which form a soft capsule by injecting and charging a predetermined amount of liquid medicine F fed from the liquid medicine feeding units 40, between the two gelatin sheets S transferred and fed from the sheet forming units 15 and cutting the gelatin sheets S, and are located at a middle part of the sheet forming units 15 for encapsulation.

Further, in order to form the two gelatin sheets S transferred and fed from the sheet forming units 15 into a capsule shape while heating and sealing the gelatin sheets S, cavities 21 engraved into a one-half shape of the capsule are provided on outer peripheries of the pair of die rolls 20 to rotate in opposite directions while facing each other and approaching each other in a state in which they are arranged to correspond to each other.

Here, blades for cutting off the encapsulated gelatin sheets S are formed at edges of the cavities 21.

Further, a hole for pulling the gelatin sheet S through vacuum absorption (negative pressure) or a suction operation or allowing a pocket to be naturally formed by a pressure at the moment when the liquid medicine F is ejected from the nozzle 31 is formed at a center of a bottom surface of each of the cavities 21.

Further, the wedge 30 has a wedge shape having symmetrically concave surfaces on opposite sides to inject the liquid medicine F between the gelatin sheets S transferred and fed between the pair of die rolls 20 and is located above the pair of die rolls 20, and a plurality of nozzles 31 for receiving the liquid medicine F from the liquid medicine feeding unit 40 and injecting the liquid medicine F between the gelatin sheets S which are to be encapsulated are arranged on the curved surfaces of the wedge 30, which are concavely formed to face the outer upper sides of the die rolls 20.

Moreover, liquid medicine feeding holes for connecting the nozzles 31 and the liquid medicine feeding unit 40 are formed on the upper surface of the wedge 30, and heaters 32 for heating the gelatin sheets S to a predetermined temperature and a second temperature sensor 34 for detecting the temperature of the wedge 30 are embedded in the wedge 30.

That is, the heaters 32 heats the wedge 30 under the control of the controller 50 and transfers heat that is necessary for heat-sealing to the gelatin sheets S.

In particular, elliptical shapes in which recesses 33 is formed to be long in the widthwise direction of the cavities 21 are formed at edges of the liquid medicine ejected parts of the nozzles 31 of the wedge 30 such that the pressure of the liquid medicine F is lowered and the liquid medicine F is injected toward one cavity 21 and another cavity 21 that is adjacent to the one cavity 21 at the same time.

Here, the wedge 30 has a shape which surrounds at least two fifths of the upper sides of the pair of die rolls 20, and it is preferable that the nozzles 31 are located within an angle range of 100 to 175 degrees or a plurality of nozzles 31 are arranged.

That is, the area of the wedge 30, which surrounds the upper sides of the pair of die rolls 20 may become larger as the formation locations of the nozzles 31 become more distant from the locations of the encapsulation, and the rotational speed of the die rolls 20 may be doubled according to the formation locations of the nozzles 31.

For example, if the nozzles 31 are formed at locations of 100 degrees with respect to the rotational shafts of the die rolls 20 having a diameter of 150 mm, the speed is increased by about eight times as compared with the existing technology, if the nozzles 31 are formed at locations of 160 degrees, the speed is increased by about 12 times as compared with the existing technology, and if the nozzles 31 are formed at locations of 175 degrees, the rotational speed of the die rolls 20 is about 26 RPM/min, which is increased by 13 times or more as compared with the existing technology in which the nozzles are formed at locations of 7.645 degrees and the rotational speed of the die rolls is 2 RPM/min.

The liquid medicine feeding unit 40 is a part which feeds the liquid medicine between the gelatin sheets S, which are to be encapsulated, and is located above the wedge 30 to feed the liquid medicine toward the nozzles 31 of the wedge 30 at a predetermined cycle (timing).

The liquid medicine feeding unit 40 may employ a general structure including a hopper, a plunger type pump, and a tube assembly.

The controller 50 generally controls an operation of the forming machine, and in particular, adjusts the heating temperature of the wedge 30 by controlling the heaters 32 according to the detected moisture content of the moisture sensor 16 and the detected temperature of the temperature sensor 34.

For example, the controller 50 may control such that a difference value from a reference of 25% is calculated if the content of moisture in the gelatin solution on the sheet forming units 15 is less than 25% and the temperatures of the heaters 32 of the wedge 30 are decreased by the calculated difference value, and a difference value from a reference of 45% is calculated when the content of moisture is more than 45% and the temperatures of the heaters 32 of the wedge 30 are increased by the calculated difference value.

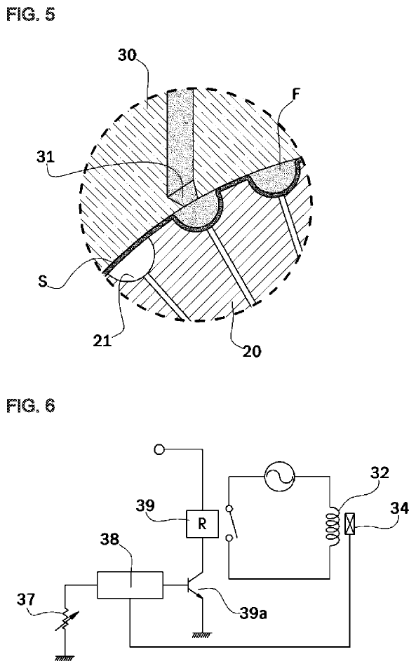

Further, as illustrated in FIG. 5, the controller 50 may include a variable adjustor 37 for arbitrarily setting the temperature of the heaters 32, a microcomputer 38 configured to compare signals of the temperature sensor 34 and the variable adjustor 37 and interrupt supply of a power source, a relay 39 installed on an electric wire for supplying a power source to control an on/off operation of the applied power source, and a transistor 39a configured to drive the relay 39.

That is, the microcomputer 38 maintains a temperature set by the variable adjustor 37 by recognizing the temperature set by the variable adjustor 37 and receiving a signal of the temperature sensor 34, and comparing the recognized temperature and the signal to control the power source applied to the heater 32, and the microcomputer 38 interrupts the power source by sending a signal to the transistor 39a to turn off the relay 39 when the temperature detected by the temperature sensor 34 is higher than the temperature set by the variable adjustor 37, and consistently applies the power source to the heaters 32 of the wedge 30 to generate heat by sending a signal to the transistor 39a to maintain the relay 39 in an on state when the temperature detected by the temperature sensor 34 is lower than the temperature set by the variable adjustor 37.

Meanwhile, it may be controlled such that the rotational speed of the die rolls 20 is increased if the content of the moisture in the gelatin solution on the sheet forming units 15 is less than 25% and the rotational speed of the die rolls 20 is decreased if the content of the moisture is more than 45%, but in this case, the production speed is inevitably remarkably decreased.

The preheating units 60 are provided between the sheet forming units 15 and the wedge 30 to send the gelatin sheets S transferred and fed from the sheet forming units 15 between the pair of die rolls 20 and the wedge 30 after preheating the gelatin sheets S in advance in a stable state without any abrupt change in temperature.

Further, a heater (not illustrated) for heating the gelatin sheet S to a predetermined temperature and a temperature sensor (not illustrated) for detecting the temperature are embedded in each of the preheating units 60.

The main operations and operational principles of the high-speed soft capsule forming machine according to the second embodiment of the present invention will be described below.

First, as illustrated in FIGS. 12 to 14, two gelatin sheets S are fed between the pair of die rolls 20 and the liquid medicine F is continuously ejected at a specific time interval through the two nozzles 31 of the wedge 30 located above the pair of die rolls 20 so that the liquid medicine F is injected between the two gelatin sheets S inserted between the die rolls 20. At the same time, the wedge 30 increases bonding force by heating the gelatin sheets S with the heaters 32 and dissolving the gelatin sheets S with the heat, and the pair of left and right die rolls 20 heat-seals the gelatin sheets S with a pressure formed when the gelatin sheets S pass through the minimum distance portion between the pair of die rolls 20. Then, the gelatin sheets S are cut to have a capsule shape as the blades (not illustrated) formed at edges of the cavities 21 of the die rolls 20 are engaged with each other.

Moreover, mangle rollers are disposed on the lower side of the die rolls 20, which have completely formed a capsule so that the remaining gelatin sheets (scrap sheets) that are left after the capsule is cut off are pulled to the lower side and at the same time, the soft capsule and the remaining gelatin sheets are separated from the gelatin sheets by release rollers, and only the soft capsule is retrieved via a shooter disposed on the front side. Then, the rotational speed of the mangle rollers is adjusted such that the remaining gelatin sheets are neither torn while being fed nor wound into the die rolls 20.

In this process, even if an error is caused in the operation times and intervals of the nozzles 31 and the die rolls 20 due to a mistake of the operator, the liquid medicine may be naturally injected into the pockets (cavities) of the gelatin sheets (S) in a state in which the pressure of the liquid medicine is decreased by the recesses 33.

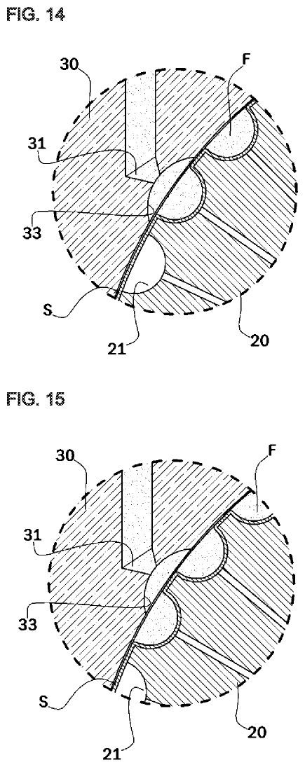

That is, as illustrated in FIG. 14, because the elliptical recesses 33 that are concavely recessed at the edges of the liquid medicine ejection parts of the nozzles 31 are formed to have a width that is larger than the width of one cavity 21 in the widthwise direction (the rotational direction of the die rolls) of the cavities 21, the liquid medicine F primarily filled in the recess 33 may be naturally injected into one cavities 21 by an ejection pressure of the liquid medicine F when the liquid medicine F is ejected if the ejection location of the nozzle 31 is normal.

Further, as illustrated in FIG. 15, because the liquid medicine F, which maintains a state in which the liquid medicine F is primarily filled in the recess 33, is ejected toward one cavity 21 and another cavity 21 that is adjacent to the one cavity 21 at the same time in a state in which the pressure of the liquid medicine F is decreased by the recess 33, the liquid medicine F can be smoothly injected into the one cavity 21 and the second cavity 21 that is adjacent to the one cavity 21 even if an injection position error of the nozzles 31 for the cavities 21 is generated.

Accordingly, because a defect at a joining part of the capsule C, which occurs when the liquid medicine F is directly ejected to the joining part due to an ejection position error of the nozzle 31 and a loss of the material can be minimized and high-speed forming is possible as well, productivity can be remarkably improved.

Further, because a change of the physical properties of the gelatin sheets S can be prevented by controlling the heating temperatures of the heaters 32 and the preheating units 60 embedded in the wedge according to the content of the moisture in the gelatin solution to heat the gelatin sheets S in a stable state without any abrupt change in temperature, defect rate can be minimized while productivity is remarkably increased, and the capsule forming machine can be used in general purpose regardless of viscosity, temperature, drying degree (the degree of moisture that is necessary to form the gelatin sheets into a capsule and maintain the encapsulation) due to the material and kind of the gelatin.

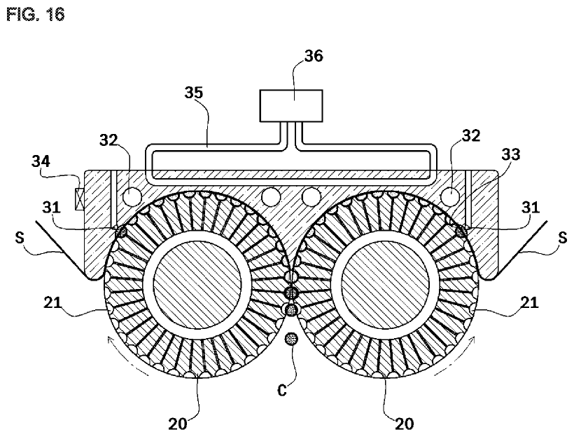

Meanwhile, as illustrated in FIG. 16, like the high-speed soft capsule forming machine according to the second embodiment of the present invention, another high-speed soft capsule forming machine according to the second embodiment of the present invention includes sheet forming units 15, a pair of die rolls 20, a wedge 30, a liquid medicine feeding unit 40, a controller 50, and a preheating unit 60.

In particular, a water jacket 35 is formed on the outer side of the wedge 30 to circulate the cooling water (refrigerant), the cooling water in the interior of the jacket 35 circulates through a cooling water inlet and a cooling water outlet of the water jacket 35, and a cooler 36 for cooling the cooling water (refrigerant), which has circulated in the interior of the water jacket 35 and has absorbed heat, and feeding the cooling water to the water jacket 35 is provided.

Here, the cooler 36 may employ a structure which realizes a general freezing cycle including a compressor for compressing a refrigerant, a condenser for condensing the refrigerant, a pressure reducer for reducing the pressure of the refrigerant, and an evaporation pipe for exchanging heat with the surroundings by evaporating the refrigerant.

Further, the cooler 36 may employ a thermoelectric element, such as a Peltier element, which uses a thermoelectric and cold energy effect from a thermoelectric semiconductor.

Further, the controller 50 can rapidly decrease the temperature of the wedge 30 by controlling the cooler 36 if a detected temperature of the temperature sensor 34, which detects the heating temperature of the wedge 30, is a normal value or more.

Accordingly, because the heating temperature of the wedge 30 may be decreased much more promptly and stably, defect rate can be minimized and operation stop time can be decreased as well.

Here, the elements of the second soft capsule forming machine according to the second embodiment of the present invention, which have the same or similar operational effects to the second embodiment are denoted by the same reference numerals, and a detailed repeated description thereof will be omitted.

Meanwhile, the present invention is not limited to the above-described embodiments and the accompanying drawings, and it is apparent to an ordinary person in the art to which the present invention pertains that the present invention may be variously modified and applied to those which are not exemplified herein without departing from the technical spirit of the present invention and the elements may be replaced and may be changed to other embodiments to be widely applied.

Therefore, the contents related to the modifications and applications of the features of the present invention have to be construed to be included in the technical spirit and the range of the present invention.

### INDUSTRIAL APPLICABILITY

According to the present invention, because the gelatin sheets may be fed in a stable state without any abrupt change in temperature in a process of making various liquid medicines into a form of a soft capsule together with gelatin sheets and the liquid medicine may be injected naturally and stably into the pockets (cavities) of the gelatin sheets, a soft capsule of an excellent quality can be formed at a high speed by minimizing the defects of the joining and sealing part due to an injection position error of the nozzles and the loss of the material.

1. A high-speed soft capsule forming machine comprising:
sheet forming units configured to coat a gelatin solution on cooling drums, which are rotating, to form two gelatin sheets;
a pair of die rolls having cavities engraved into a one-half shape of the capsule provided on outer peripheries of the pair of die rolls so as to rotate in opposite directions while facing each other and approaching each other in a state in which the cavities are arranged to correspond to each other, in order to form the gelatin sheets into a capsule shape while heat-sealing the gelatin sheets;
a wedge which is located above the pair of die rolls, in which one or more rows of nozzles for receiving liquid medicine from a liquid medicine feeding unit and injecting the liquid medicine between the gelatin sheets inserted into the cavities of the pair of die rolls are symmetrically formed, and in which heaters for heating the gelatin sheets to a predetermined temperature are embedded in the wedge; and
a controller configured to control rotational speeds of the pair of die rolls and the cooling drums in proportion to a preset angle from the center of the pair of die rolls to the nozzles formed in the wedge, to control temperatures of the wedge and the cooling drums in inverse proportion to the preset angle, and to control an interval of operations of the nozzles for injection of the liquid medicine in proportion to a preset number of rows of the arranged nozzles to adjust forming speed,
wherein the controller controls such that whenever an angle from a starting point, which is a section from the centers of the pair of die rolls 20 to a part in which the gelatin sheets S are joined and sealed, to the nozzles formed in the wedge increases by 30° from a reference angle (30°), the rotational speed of the pair of die rolls increases by 4 to 6 RPM/min from a reference rotational speed (4 to 6 RPM/min), a surface temperature of the wedge increases by 1 to 2° C. from a reference temperature (38 to 42° C.), the rotational speed of the cooling drums increases by 1.2 to 1.8 RPM/min from a reference rotational speed (1.2 to 1.8 RPM/min), a surface temperature of the cooling drums decreases by 1° C. from a reference temperature (20 to 18° C.), and the interval for the operations of the nozzles for one dose of liquid medicine further increases by 3 times per second from a reference number of times (3 times per second), and when the number of rows of the nozzles increases by 1 row from a reference row (1 row) formed in the reference angle, the number of times decreases by a value obtained by dividing the reference number of times for the reference angles by the increased number of rows.

2. The high-speed soft capsule forming machine of claim 1, wherein one or more rows of nozzles are arranged along concave surfaces of lower portions of the wedge formed to correspond to the pair of die rolls, and are symmetrically arranged on opposite sides of the wedge to be located within an angle range of 7° to 168° from the center of one die roll.

3. The high-speed soft capsule forming machine of claim 1, wherein curved protrusions which heat the gelatin sheets while contacting the gelatin sheets protrude convexly from the opposite sides of the wedge.

4. A high-speed soft capsule forming machine comprising:
sheet forming units configured to form two gelatin sheets from a gelatin solution;
a pair of die rolls having cavities engraved into a one-half shape of the capsule provided on outer peripheries of the pair of die rolls so as to rotate in opposite directions while facing each other and approaching each other in a state in which the cavities are arranged to correspond to each other, in order to form the gelatin sheets into a capsule shape while heat-sealing the gelatin sheets; and
a wedge which is located on the upper side of the pair of die rolls, in which a pair of nozzles for receiving liquid medicine from a liquid medicine feeding unit and injecting the liquid medicine between the gelatin sheets, and in which heaters for heating the gelatin sheets to a predetermined temperature and a temperature sensor for detecting temperature are embedded,
wherein a recess is formed long in a widthwise direction of the cavities to eject the liquid medicine to one cavity and another adjacent cavity at the same time after decreasing an ejection pressure of the liquid medicine, at an edge of a part of the wedge, in which the nozzle ejects the liquid medicine.

5. The high-speed soft capsule forming machine of claim 4, wherein a moisture sensor for electrically detecting a content of moisture in the gelatin solution is provided on each of the sheet forming unit, and a controller for adjusting a heating temperature of the wedge by controlling the heaters depending on a detected moisture content of the moisture sensor and a detected temperature of the temperature sensor is provided.

6. The high-speed soft capsule forming machine of claim 5, further comprising:
a water jacket formed on the outer side of the wedge to circulate cooling water;
a cooler configured to circulate the cooling water in the water jacket through a cooling water inlet and a cooling water outlet of the water jacket and cool the cooling water which has absorbed heat while circulating the interior of the water jacket; and
a controller configured to, if the detected temperature of the temperature sensor is a normal value or more, rapidly decrease the temperature of the wedge by controlling the cooler.

7. The high-speed soft capsule forming machine of claim 4 or 5, further comprising:
preheating units configured to preheat the gelatin sheets into a stable state without any abrupt change of temperature, between the sheet forming units and the wedge.

8. The high-speed soft capsule forming machine of claim 7, wherein a moisture sensor for electrically detecting a content of moisture in the gelatin solution is provided on each of the sheet forming unit, and a controller for adjusting heating temperatures of the wedge and the preheating units by controlling the heaters of the wedge and the preheating units depending on a detected moisture content of the moisture sensor is provided.

9. The high-speed soft capsule forming machine of claim 5, wherein the controller controls such that a difference value from a reference of 25% is calculated if the content of moisture in the gelatin solution on the sheet forming units is less than 25% and the temperatures of the heaters of the wedge are decreased by the calculated difference value, and a difference value from a reference of 45% is calculated when the content of moisture is more than 45% and the temperatures of the heaters of the wedge are increased by the calculated difference value.
